(19) United States
(12) Patent Application Publication
(10) Pub. No.: US 2026/0157758 A1
(43) Pub. Date: Jun. 11, 2026

(54) DELIVERY AND DETACHMENT SYSTEM FOR AN IMPLANTABLE INTRAVASCULAR TREATMENT DEVICE

(71) Applicant: DEPUY SYNTHES PRODUCTS, INC., RAYNHAM, MA (US)

(72) Inventors: LACEY GOROCHOW, MIAMI, FL (US); DANIEL SOLAUN, MIAMI, FL (US); THOMAS GALLERANI, MIAMI, FL (US); RAVI ATMAKURU, RAYNHAM, MA (US)

(21) Appl. No.: 19/180,641

(22) Filed: Apr. 16, 2025

Related U.S. Application Data
(60) Provisional application No. 63/647,693, filed on May 15, 2024.

(51) Int. Cl. A61B 17/12; A61B 17/00; A61M 25/00

(52) U.S. Cl. CPC A61B 17/1214; A61B 2017/00367; A61B 2017/12054; A61M 2025/0004; A61M 25/0068

(57) ABSTRACT

A delivery and detachment system includes an outer tube, an inner tube, and a pull wire. The inner tube is disposed radially within the outer tube. The inner diameter of the outer tube is greater than the outer diameter of the inner tube. The inner tube has a first slot and has a second outer diameter at the first slot. The second outer diameter of the inner tube is greater than the inner diameter of the outer tube. A portion of the outer circumferential surface of the inner tube adjacent to the first slot is in contact with and has a friction fit with the inner circumferential surface of the outer tube and is configured to prevent unintended movement of the inner tube and the pull wire with respect to the outer tube and thereby prevent premature implant deployment.

### CROSS-REFERENCE TO RELATED APPLICATIONS

The present application claims the benefit of U.S. Provisional Application No. 63/647,693, filed May 15, 2024, the disclosure of which is hereby incorporated by reference herein.

### FIELD

The present disclosure relates to an intravascular treatment device and more particularly, to improved delivery systems for embolic implants that prevent premature implant deployment.

### BACKGROUND

Numerous intravascular implant devices are known in the field. Many are deployed mechanically, via systems that combine one or more catheters and wires for delivery. Examples of implants that can be delivered mechanically include embolic elements, intrasaccular devices, stents, grafts, drug delivery implants, flow diverters, filters, stimulation leads, sensing leads, or other implantable structures delivered through a microcatheter. Some obstetric and gastrointestinal implants may also be implanted via similar systems that combine one or more catheters and wires. Devices that may be released or deployed by mechanical means vary greatly in design but can employ a similar delivery catheter and wire system. Many such catheter-based delivery systems include a wire for retention of the implant in the catheter until the time for release of the device. These systems are then actuated by retracting or pulling the wire relative to the catheter. Such a wire is referred to herein as a “pull wire”.

One issue with current catheter-based delivery systems is premature detachment of the implantable device. Premature detachment occurs when the implant is detached from the delivery system before reaching the treatment site. This may occur due to the tortuosity experienced by the delivery system as it passes through the vasculature of the patient, which can cause an increase in friction between the “pull wire” and the delivery system causing the pull wire to move proximally while the delivery system is moving distally.

Accordingly, there is a need for an improved implant delivery system that prevents premature detachment of the implant as it is delivered through tortuous vasculature. This disclosure is directed to this and other considerations.

### SUMMARY

Disclosed herein are various exemplary systems, devices, and methods for inhibiting premature implant deployment. The delivery member can include a delivery and detachment system for an implantable intravascular treatment device. The system includes an outer tube, an inner tube, and a pull wire. The outer tube has a proximal end, a distal end, an inner diameter, an inner circumferential surface, and a lumen extending axially therethrough from the proximal end to the distal end. The inner tube has a proximal end, a distal end, a first outer diameter, an outer circumferential surface, and a lumen extending axially therethrough from the proximal end to the distal end. The inner tube is disposed radially within the outer tube at the proximal end of the outer tube. The inner diameter of the outer tube is greater than the outer diameter of the inner tube. The pull wire is positioned within the lumen of the outer tube and the lumen of the inner tube. The pull wire has a proximal end, and a distal end. The proximal end of the pull wire is positioned within the lumen of the inner tube. The proximal end of the pull wire is fixedly connected to the inner tube. The inner tube has a first slot proximate the proximal end of the pull wire. The inner tube has a second outer diameter at the first slot. The second outer diameter of the inner tube is greater than the inner diameter of the outer tube. A portion of the outer circumferential surface of the inner tube adjacent to the first slot is in contact with and has a friction fit with the inner circumferential surface of the outer tube and is configured to prevent unintended movement of the inner tube and the pull wire with respect to the outer tube and thereby prevent premature implant deployment.

### BRIEF DESCRIPTION OF THE DRAWINGS

The above and further aspects of this disclosure are further discussed with reference to the following description in conjunction with the accompanying drawings, in which like numerals indicate like structural elements and features in various figures. The drawings are not necessarily to scale, emphasis instead being placed upon illustrating principles of the disclosure. The figures depict one or more implementations of the inventive devices, by way of example only, not by way of limitation.

FIG. 1 is a plan view, with parts shown in cross-section, of the delivery and detachment system according to aspects of the present disclosure.

FIG. 2A is a plan view of an inner tube in accordance with the present disclosure with parts broken away.

FIG. 2B is a plan view of the inner tube of FIG. 2A showing the inner tube with its outer diameter expanded at a slot within the inner tube.

FIG. 2C is a cross-sectional view taken along lines 2C-2C of FIG. 2B.

FIG. 3A is a plan view of an inner tube in accordance with another aspect of the present disclosure, shown with parts broken away.

FIG. 3B is a plan view of the inner tube of FIG. 3A showing the inner tube with its outer diameter expanded at a slot within the inner tube.

FIG. 3C is a partial cross-sectional view showing the inner tube of FIG. 3B within an outer tube.

FIG. 4A is a plan view of an inner tube in accordance with another aspect of the present disclosure.

FIG. 4B is a partial cross-sectional view showing the inner tube of FIG. 4A within an outer tube.

FIG. 5A is a plan view of an inner tube in accordance with another aspect of the present disclosure.

FIG. 5B is a partial cross-sectional view showing the inner tube of FIG. 5A within an outer tube.

FIG. 6A is a plan view of an inner tube in accordance with another aspect of the present disclosure.

FIG. 6B is a partial cross-sectional view showing the inner tube of FIG. 6A within an outer tube.

FIG. 7A is a plan view of an inner tube in accordance with another aspect of the present disclosure.

FIG. 7B is a partial cross-sectional view showing the inner tube of FIG. 7A within an outer tube.

### DETAILED DESCRIPTION

As used herein, the terms “about” or “approximately” for any numerical values or ranges indicate a suitable dimensional tolerance that allows the part or collection of components to function for its intended purpose as described herein. More specifically, “about” or “approximately” may refer to the range of values ±20% of the recited value, e.g. “about 90%” may refer to the range of values from 71% to 99%.

As used herein, the terms “tubular” and “tube” are to be construed broadly and are not limited to a structure that is a right cylinder or strictly circumferential in cross-section or of a uniform cross-section throughout its length. For example, a tubular structure or system is generally illustrated as a substantially right cylindrical structure. However, the tubular system may have a tapered or curved outer surface without departing from the scope of the present disclosure.

Documents incorporated by reference in the present patent application are to be considered an integral part of the application except that to the extent any terms are defined in these incorporated documents in a manner that conflicts with the definitions made explicitly or implicitly in the present specification, only the definitions in the present specification should be considered.

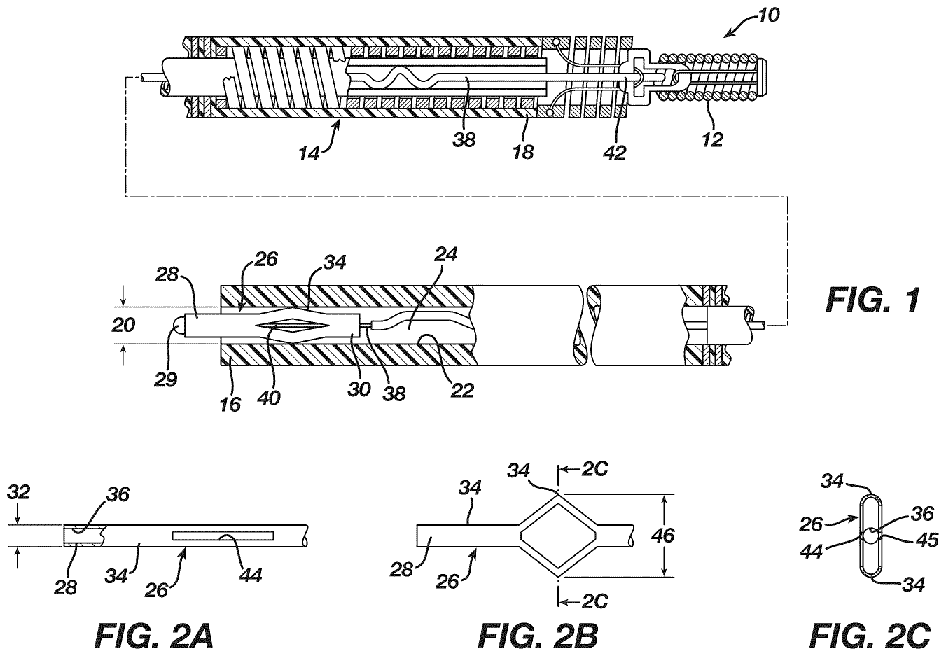

Referring now to FIGS. 1-2C , a delivery and detachment system 10 for an implantable intravascular treatment device 12 is illustrated. Device 12 may be, for example, embolic coil elements, intrasaccular devices, stents, grafts, drug delivery implants, flow diverters, filters, stimulation leads, sensing leads, or other implantable structures delivered through a microcatheter. Some obstetric and gastrointestinal implants may also be implanted via similar systems that combine one or more catheters and wires. Delivery and detachment system 10 includes an outer tube 14, an inner tube 26, and a pull wire 38. Outer tube 14 has a proximal end 16, a distal end 18, an inner diameter 20, an inner circumferential surface 22, and a lumen 24 extending axially therethrough from the proximal end 16 to the distal end 18. Inner tube 26 comprises a proximal end 28, a distal end 30, a first outer diameter 32, an outer circumferential surface 34, and a lumen 36 extending axially therethrough from the proximal end 28 to the distal end 30. Inner tube 26 is disposed radially within the outer tube 14 at the proximal end 16 of the outer tube 14. The inner diameter 20 of the outer tube 14 is greater than the outer diameter 32 of the inner tube 26.

Pull wire 38 is positioned within the lumen 24 of the outer tube 14 and the lumen 36 of the inner tube 26. Pull wire 38 has a proximal end 40, and a distal end 42. The proximal end 40 of the pull wire 38 is positioned within the lumen 36 of the inner tube 26. The proximal end 40 of pull wire 38 is fixedly connected to the inner tube 26 at joint 29. The pull wire distal end 42 is releasably connected to an implantable intravascular treatment device 12. In one example, the implantable intravascular treatment device 12 is an embolic coil that is configured to treat an aneurysm.

Inner tube 26 has a first slot 44 proximate the proximal end 40 of the pull wire 38. Inner tube 26 has a second outer diameter 46 at the first slot 44. See, for example, FIG. 2B. The second outer diameter 46 of the inner tube 26 is greater than the inner diameter 20 of the outer tube 14. A portion of the outer circumferential surface 34 of the inner tube 26 adjacent to the first slot 44 is in contact with and has a friction fit with the inner circumferential surface 22 of the outer tube 14. This friction fit is configured to prevent unintended movement of the inner tube 26 and the pull wire 38 with respect to the outer tube 14.

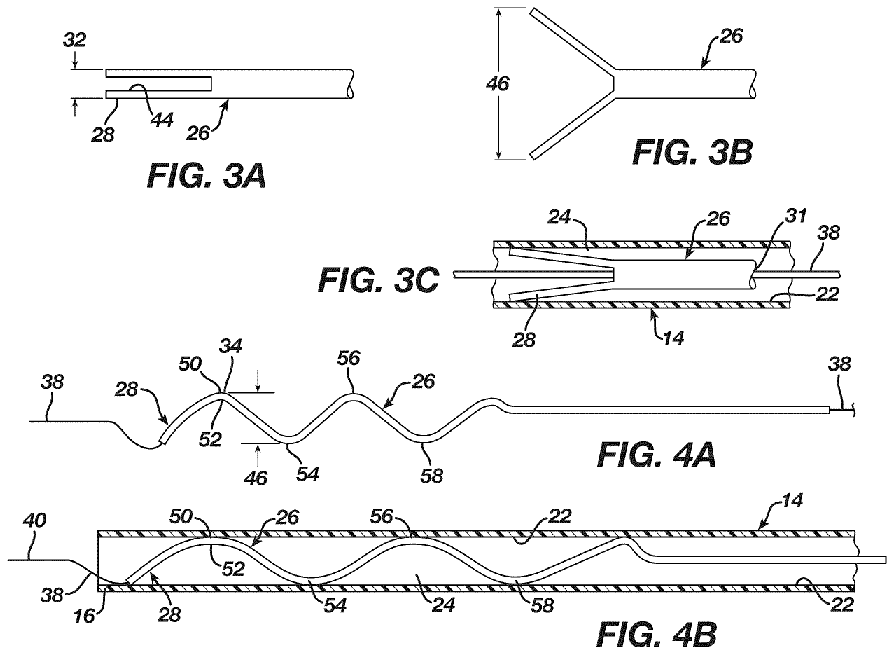

As shown in FIGS. 2A and 2B, for example, the first slot 44 is spaced from the proximal end 28 of the inner tube 26. Alternatively, as shown in FIGS. 3A-3C, the first slot 44 can extend to the proximal end 28 of the inner tube 26. In the example shown in FIGS. 2A-2C, the proximal end 28 of the inner tube 26 extends proximally beyond the proximal end 16 of the outer tube 14. As shown in FIG. 1, the proximal end 40 of the pull wire 38 is fixedly connected to the proximal end 28 of the inner tube 26 at joint 29. Joint 29 can be, for example, a welded joint, a soldered joint, or a glued joint. As shown in FIG. 2C, inner tube 26 has a second slot 45 proximate the proximal end 40 of the pull wire 38. Second slot 45 is approximately diametrically opposite to the first slot 44. After forming slots 44 and 45, inner tube 26 can be compressed about the slots to form the shape shown in FIG. 2B. Inner tube 26 can then be heat treated to maintain the shape shown in FIG. 2B. Thereafter, inner tube 26 can then be placed within outer tube 14 as shown in FIG. 1 such that the proximal end 28 of the inner tube 26 is proximal of the proximal end 16 of the outer tube 14. The enlarged outer diameter portion of inner tube 26 can be compressed to fit within outer tube 14. The enlarged outer diameter portion of inner tube 26 is now in a friction fit with outer tube 14 to prevent unintended movement of the inner tube 26 and the pull wire 38 with respect to the outer tube 14. When the implant 12 is positioned at the desired treatment site within the vasculature, a user can then pull on the proximal end of inner tube 26 and pull the inner tube and pull wire 38 in the proximal direction with sufficient force to overcome the friction fit between the inner tube and the outer tube to thereby release or detach implant 12 within the vasculature. The friction fit between the inner tube 26 and outer tube 14 prevents inadvertent movement of the pull wire 38 while the implant 12 is moving within the vasculature to the treatment site.

In one example, inner tube 26 can be made from a suitable material, such as, for example, a metal or plastic that can be strained to at least a larger diameter than the outer tube 14. In one example, the inner tube can be made from Nitinol (“NiTi”). A nitinol inner tube can be heat treated to its preferred shape as shown in FIG. 2B. In the heat treating process, the inner tube can be heated to approximately 500-550° C. for 5-15 minutes and then the inner tube 14 can be quenched in cold water. The outer tube 14 can also be made from metal or plastic, such as, for example, stainless steel.

In one example, the inner diameter of the outer tube is approximately 0.009 inches. The outer diameter of the inner tube 26 is approximately 0.008 inches. However, the unrestrained outer diameter 46 of the inner tube 26 at slot 44, 45 is approximately 0.0095 inches or larger. The inner diameter 20 of the outer tube can be as low as 0.004 inches, the outer diameter of the inner tube can be as low as 0.003 inches, the outer diameter 46 of the inner tube at slot 44, 45 can be as low as 0.0035 inches or larger. A current limitation on the larger sizes is that the outer tube 14 should fit within a 0.0165-inch microcatheter.

Referring now to FIGS. 3A-3C, another example of the present disclosure is illustrated. First slot 44 extends to the proximal end 28 of the inner tube 26. A second slot (not shown) is also formed in the proximal end 28 of inner tube 26 approximately diametrically opposite of slot 44. In the example shown in FIGS. 3A-3C, the proximal end 28 of the inner tube 26 is disposed within the lumen 24 of the outer tube 14. In this example, the pull wire 38 is fixedly connected to the inner tube 26 at a joint 31 that is proximal of slot 44. Joint 31 can be, for example, a welded joint, a soldered joint, or a glued joint. As shown in FIG. 3B, inner tube 26 distal end can be flared radially outwardly to the position shown in FIG. 3B. Inner tube 26 can then be heat treated to maintain the shape shown in FIG. 3B. Thereafter, inner tube 26 can then be placed within outer tube 14 as shown in FIG. 3C. The enlarged outer diameter portion of inner tube 26 can be compressed to fit within outer tube 14. The enlarged outer diameter portion of inner tube 26 is now in a friction fit with outer tube 14 to prevent unintended movement of the inner tube 26 and the pull wire 38 with respect to the outer tube 14. When the implant 12 is positioned at the desired treatment site within the vasculature, a user can then pull on the proximal end of pull wire, which is proximal of the proximal ends of the inner tube 26 and outer tube 14, and pull the inner tube 26 and pull wire 38 in the proximal direction with sufficient force to overcome the friction fit between the inner tube and the outer tube to thereby release or detach implant 12 within the vasculature. The friction fit between the inner tube 26 and outer tube 14 prevents inadvertent movement of the pull wire 38 while the implant 12 is moving within the vasculature to the treatment site.

Referring now to FIGS. 4A and 4B, another example of the present disclosure is illustrated. In this example, outer tube 14 and inner tube 26 are like the examples shown in FIGS. 1-2C and 3A-3C and only the differences from these other examples will be described for the sake of brevity in the disclosure. A pull wire 38 is positioned within the lumen 24 of the outer tube 14 and the lumen 24 of the inner tube 26 in a manner like the examples shown in FIGS. 1-2C and 3A-3C. Like the example of FIGS. 3A-3C, the proximal end 40 of pull wire 38 is proximal of the proximal ends of the inner tube 26 and the outer tube 14, as shown in FIG. 4B. The proximal end portion of the pull wire 38 disposed within inner tube 26 is fixedly connected to the inner tube 26 so that the pull wire 38 and the inner tube 26 are fixedly connected to each other. A proximal end portion 28 of the inner tube 26 that is disposed within the lumen 24 of the outer tube 14 is shaped to have at least one bend 50. As shown in FIGS. 4A and 4B, there are four bends 52, 54, 56, 58 in one example. There could be as few as one bend or more than four. But in all examples, the at least one bend 50 creates a larger effective outer diameter 46 than the inner diameter 20 of the outer tube 14 such that a portion of the outer circumferential surface 34 of the inner tube proximal end portion 28 is in contact with and has a friction fit with the inner circumferential surface 22 of the outer tube 14 and is configured to prevent unintended movement of the inner tube 26 and the pull wire 38 with respect to the outer tube 14. As shown, the bends 50 create a sinusoidal curve. When the inner tube 26 is within the outer tube 14, the sinusoidal curve created by the bends 52, 54, 56 and 58 are compressed such that portions of the outer circumferential surface of the inner tube proximal end portion are in contact with and have a friction fit with the inner circumferential surface of the outer tube. Also, as shown in in FIG. 4B, the proximal end 40 of pull wire 38 extends proximally beyond the proximal end 16 of the outer tube 14.

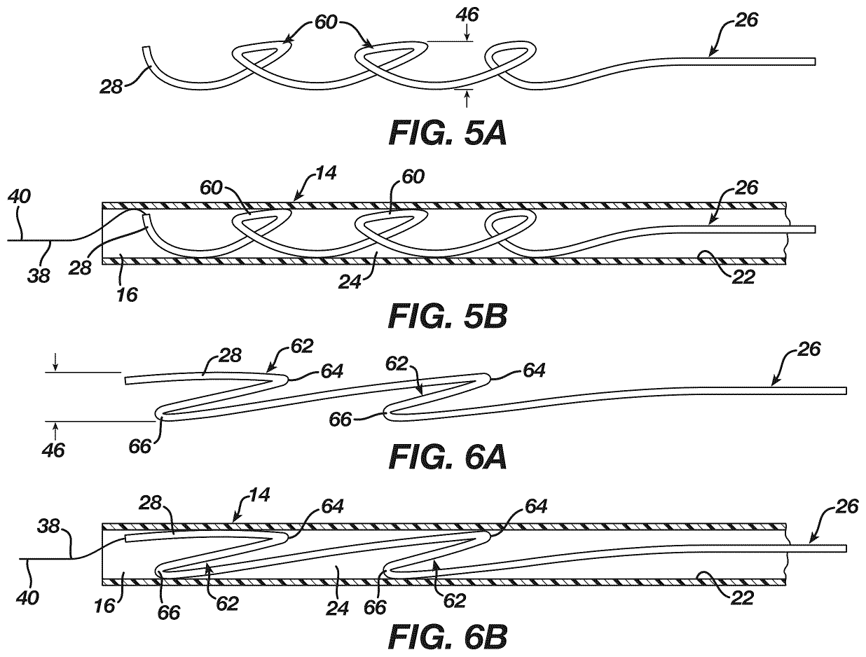

Referring now to FIGS. 5A and 5B, another example of the present disclosure is illustrated. In this example, outer tube 14 and inner tube 26 are like the examples shown in FIGS. 1-2C, 3A-3C, and 4A-4B and only the differences from these other examples will be described for the sake of brevity in the disclosure. A proximal end portion 28 of the inner tube 26 that is disposed within the lumen 24 of the outer tube 14 is shaped to have at least one loop-the-loop 60. As shown in FIGS. 5A and 5B, there are three loop-the-loops 60 in one example. There could be as few as one loop-the-loop, two loop-the-loops, or more than four. But in all examples, the at least one loop-the-loop 60 creates a larger effective outer diameter 46 than the inner diameter 20 of the outer tube 14 such that, when the proximal end portion 28 of the inner tube is disposed within the lumen 24 of the outer tube 14, a portion of the outer circumferential surface 34 of the inner tube proximal end portion 28 is in contact with and has a friction fit with the inner circumferential surface 22 of the outer tube 14 and is configured to prevent unintended movement of the inner tube 26 and the pull wire 38 with respect to the outer tube 14. As shown, the loop-the-loops 60 create a series of loops. The loop-the-loop shape ascends upward and curves back proximally on itself to form a complete loop, before descending back to its original distal direction. When the inner tube 26 is within the outer tube 14, the loops 60 are compressed such that portions of the outer circumferential surface of the inner tube proximal end portion are in contact with and have a friction fit with the inner circumferential surface of the outer tube. Also, the proximal end 40 of pull wire 38 extends proximally beyond the proximal end 16 of the outer tube 14.

Referring now to FIGS. 6A and 6B, another example of the present disclosure is illustrated. In this example, outer tube 14 and inner tube 26 are like the examples shown in FIGS. 1-2C, 3A-3C, 4A-4B, and 5A-5B, and only the differences from these other examples will be described for the sake of brevity in the disclosure. A proximal end portion 28 of the inner tube 26 that is disposed within the lumen 24 of the outer tube 14 is shaped to have at least one zigzag 62. As shown in FIGS. 6A and 6B, there are two zigzags 62 in one example. There could be as few as one zigzag, two zigzags, or more than three. But in all examples, the at least one zigzag 62 creates a larger effective outer diameter 46 than the inner diameter 20 of the outer tube 14 such that, when the proximal end portion 28 of the inner tube is disposed within the lumen 24 of the outer tube 14, a portion of the outer circumferential surface 34 of the inner tube proximal end portion 28 is in contact with and has a friction fit with the inner circumferential surface 22 of the outer tube 14 and is configured to prevent unintended movement of the inner tube 26 and the pull wire 38 with respect to the outer tube 14. As shown, the zigzags 62 create a series of loops. The zigzag shape extends distally and then bends back proximally at a first bend 64 for a predetermined distance and then bends back distally at a second bend 66 and then extends distally beyond the first bend 64. When the inner tube 26 is within the outer tube 14, the zigzags 62 are compressed such that portions of the outer circumferential surface of the inner tube proximal end portion are in contact with and have a friction fit with the inner circumferential surface of the outer tube. Also, the proximal end 40 of pull wire 38 extends proximally beyond the proximal end 16 of the outer tube 14.

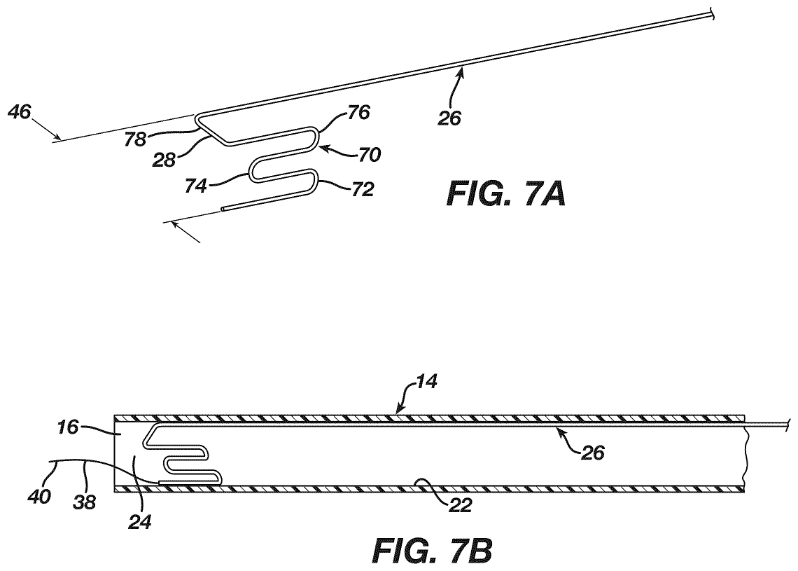

Referring now to FIGS. 7A and 7B, another example of the present disclosure is illustrated. In this example, outer tube 14 and inner tube 26 are like the examples shown in FIGS. 1-2C, 3A-3C, 4A-4B, 5A-5B, and 6A-6B, and only the differences from these other examples will be described for the sake of brevity in the disclosure. A proximal end portion 28 of the inner tube 26 that is disposed within the lumen 24 of the outer tube 14 is shaped to have at least one complex three-dimensional shape 70. As shown in FIGS. 7A and 7B, in one example, there are four 180-degree bends 72, 74, 76, 78 in the inner tube 26. There could be as few as two 180-degree bends, or more than four. The at least four 180-degree bends creates a larger effective outer diameter 46 than the inner diameter 20 of the outer tube 14 such that, when the proximal end portion 28 of the inner tube is disposed within the lumen 24 of the outer tube 14, a portion of the outer circumferential surface 34 of the inner tube proximal end portion 28 is in contact with and has a friction fit with the inner circumferential surface 22 of the outer tube 14 and is configured to prevent unintended movement of the inner tube 26 and the pull wire 38 with respect to the outer tube 14. When the inner tube 26 is within the outer tube 14, the 180-degree bends are compressed such that portions of the outer circumferential surface of the inner tube proximal end portion are in contact with and have a friction fit with the inner circumferential surface of the outer tube. Also, the proximal end 40 of pull wire 38 extends proximally beyond the proximal end 16 of the outer tube 14.

Aspects of the disclosure are also provided by the following numbered clauses:

Clause 1: A delivery and detachment system 10 for an implantable intravascular treatment device 12, the system comprising:
an outer tube 14 comprising a proximal end 16, a distal end 18, an inner diameter 20, an inner circumferential surface 22, and a lumen 24 extending axially therethrough from the proximal end 16 to the distal end 18;
an inner tube 26 comprising a proximal end 28, a distal end 30, a first outer diameter 32, an outer circumferential surface 34, and a lumen 36 extending axially therethrough from the proximal end 28 to the distal end 30, the inner tube 26 being disposed radially within the outer tube 14 at the proximal end 16 of the outer tube 14, the inner diameter 20 of the outer tube 14 being greater than the outer diameter 32 of the inner tube 26;
a pull wire 38 positioned within the lumen 36 of the outer tube 14 and the lumen 24 of the inner tube 26, the pull wire 38 comprising a proximal end 40, and a distal end 42, the proximal end 40 of the pull wire 38 being positioned within the lumen 36 of the inner tube 26, the proximal end 40 of the pull wire 38 being fixedly connected to the inner tube 26;
the inner tube 26 having a first slot 44 proximate the proximal end 40 of the pull wire 38, the inner tube 26 having a second outer diameter 46 at the first slot 44, the second outer diameter 46 of the inner tube 26 being greater than the inner diameter 20 of the outer tube 14, a portion of the outer circumferential surface 34 of the inner tube 26 adjacent to the first slot 44 being in contact with and having a friction fit with the inner circumferential surface 22 of the outer tube 14 and is configured to prevent unintended movement of the inner tube 26 and the pull wire 38 with respect to the outer tube 14.

Clause 2. The delivery and detachment system of clause 1, wherein the first slot 44 is spaced from the proximal end 28 of the inner tube 26.

Clause 3. The delivery and detachment system of clause 1, wherein the first slot 44 extends to the proximal end 28 of the inner tube 26.

Clause 4. The delivery and detachment system of clause 1, wherein the proximal end 28 of the inner tube 26 extends proximally beyond the proximal end 16 of the outer tube 14.

Clause 5. The delivery and detachment system of clause 1, wherein the proximal end 40 of the pull wire 38 being fixedly connected to the proximal end 28 of the inner tube 26.

Clause 6. The delivery and detachment system of clause 1, wherein the inner tube 26 having a second slot 45 proximate the proximal end 40 of the pull wire 38, the second slot 45 being approximately diametrically opposite of the first slot 44.

Clause 7. The delivery and detachment system of clause 1, wherein the pull wire distal end 42 being releasably connected to an implantable intravascular treatment device 12.

Clause 8. The delivery and detachment system of clause 7, wherein the implantable intravascular treatment device 12 is an embolic coil configured to treat an aneurysm.

Clause 9. A delivery and detachment system 10 for an implantable intravascular treatment device 12, the system comprising:
an outer tube 14 comprising a proximal end 16, a distal end 18, an inner diameter 20, an inner circumferential surface 22, and a lumen 24 extending axially therethrough from the proximal end 16 to the distal end 18;
an inner tube 26 comprising a proximal end 28, a distal end 30, a first outer diameter 32, an outer circumferential surface 34, and a lumen 36 extending axially therethrough from the proximal end 28 to the distal end 30, the inner tube 26 being disposed radially within the outer tube 14 at the proximal end 16 of the outer tube 14, the inner diameter 20 of the outer tube 14 being greater than the outer diameter 32 of the inner tube 26;
a pull wire 38 positioned within the lumen 24 of the outer tube 14 and the lumen 24 of the inner tube 26, the pull wire 38 comprising a proximal end 40, and a distal end 42, the proximal end 40 of the pull wire being 38 being positioned within the lumen 36 of the inner tube 26, the proximal end 40 of the pull wire 38 being fixedly connected to the inner tube 26;
the inner tube 26 having its proximal end portion 28 disposed within the lumen 24 of the outer tube 14 being shaped to have at least one bend 50 in the proximal end portion 28, the at least one bend 50 being larger than the inner diameter 20 of the outer tube 14 such that a portion of the outer circumferential surface 34 of the inner tube proximal end portion 28 being in contact with and having a friction fit with the inner circumferential surface 22 of the outer tube 14 and is configured to prevent unintended movement of the inner tube 26 and the pull wire 38 with respect to the outer tube 14.

Clause 10. The delivery and detachment system of clause 9, wherein the proximal end 40 of pull wire 38 extends proximally beyond the proximal end 16 of the outer tube 14.

Clause 11. The delivery and detachment system of clause 9, wherein the pull wire 38 being fixedly connected to the inner tube 26.

Clause 12. The delivery and detachment system of clause 9, wherein the inner tube proximal end portion 28 has at least four bends 52, 54, 56, 58 and at least four axially spaced apart locations where the outer circumferential surface of the inner tube proximal end portion is in contact with and has a friction fit with the inner circumferential surface of the outer tube.

Clause 13. The delivery and detachment system of clause 12, wherein the inner tube proximal end portion 28 has a sinusoidal shape.

Clause 14. The delivery and detachment system of clause 9, wherein the inner tube proximal end portion 28 has a loop-the-loop shape 60.

Clause 15. The delivery and detachment system of clause 14, wherein the loop-the-loop shape ascends upward and curves back proximally on itself to form a complete loop, before descending back to its original distal direction.

Clause 16. The delivery and detachment system of clause 15, wherein the loop-the-loop shape includes at least two complete loops.

Clause 17. The delivery and detachment system of clause 16, wherein the loop-the-loop shape includes at least three complete loops.

Clause 18 The delivery and detachment system of clause 10, wherein the inner tube proximal end portion 28 has a zigzag shape 62.

Clause 19. The delivery and detachment system of clause 18, wherein the zigzag shape extends distally and then bends back proximally at a first bend 64 for a predetermined distance and then bends back distally at a second bend 66 and then extends distally beyond the first bend 64.

Clause 20. The delivery and detachment system of clause 19, wherein the zigzag shape includes at least two zigzags.

Clause 21. The delivery and detachment system of clause 9, wherein the pull wire distal end 40 being releasably connected to an implantable intravascular treatment device 12.

Clause 22. The delivery and detachment system of clause 21, wherein the implantable intravascular treatment device 12 is an embolic coil configured to treat an aneurysm.

Clause 23. A method of manipulating an implant delivery system to prevent unintended detachment of the implant comprising the steps of:
providing an implantation device 10 comprising a pull wire 38, an inner tube 26, and an outer tube 14;
fixedly connecting the pull wire 38 to the inner tube 26; and
shaping the inner tube 26 so that a portion of an outer circumferential surface 34 of the inner tube 26 is in contact with and has a friction fit with an inner circumferential surface 22 of the outer tube 14 to prevent unintended movement of the inner tube 26 and the pull wire 38 with respect to the outer tube 14.

Clause 24. The method of clause 23, further comprising the step of:
forming a first slot 44 in the inner tube 26 proximate the proximal end 28 of the inner tube;
forming a second slot 45 in the inner tube 26 proximate the proximal end 28 of the inner tube 26, the second slot 45 being approximately diametrically opposite to the first slot 44;
expanding the inner tube 26 to expand the shape of the inner tube adjacent to the first slot 44 and the second slot 45 such that the inner tube 26 has a second outer diameter 46 adjacent to the first slot and the second slot 45, the second outer diameter 46 of the inner tube 26 being greater than the inner diameter 20 of the outer tube 14;
placing a portion of the outer circumferential surface 34 of the inner tube 26 adjacent to the first slot 44 and the second slot 45 in contact with and having a friction fit with the inner circumferential surface 22 of the outer tube 14 to prevent unintended movement of the inner tube 26 and the pull wire 38 with respect to the outer tube 14.

Clause 25. The method of clause 24, further comprising the step of:
heat treating the inner tube 26, after the expanding step, to maintain the shape of the inner tube in the compressed and expanded shape.

Clause 26. The method of clause 25, wherein the first slot 44 and the second slot 45 are formed in the inner tube 26 a predetermined distance from the proximal end of the inner tube.

Clause 27. The method of clause 25, wherein the first slot 44 and the second slot 45 are formed in the inner tube and each slot 44, 45 extends to the proximal end 28 of the inner tube 26.

The descriptions contained herein are examples of embodiments of the disclosure and are not intended in any way to limit the scope of the disclosure. As described herein, the disclosure contemplates many variations and modifications of a delivery and detachment system for an implantable intravascular treatment device and a method of manipulating an implant delivery system to prevent unintended detachment of an implant. Modifications and variations apparent to those having skilled in the pertinent art according to the teachings of this disclosure are intended to be within the scope of the claims which follow.

What is claimed is:

1. A delivery and detachment system for an implantable intravascular treatment device, the system comprising:
an outer tube comprising a proximal end, a distal end, an inner diameter, an inner circumferential surface, and a lumen extending axially therethrough from the proximal end to the distal end;
an inner tube comprising a proximal end, a distal end, a first outer diameter, an outer circumferential surface, and a lumen extending axially therethrough from the proximal end to the distal end, the inner tube being disposed radially within the outer tube at the proximal end of the outer tube, the inner diameter of the outer tube being greater than the outer diameter of the inner tube;
a pull wire positioned within the lumen of the outer tube and the lumen of the inner tube, the pull wire comprising a proximal end, and a distal end, the proximal end of the pull wire being positioned within the lumen of the inner tube, the proximal end of the pull wire being fixedly connected to the inner tube;
the inner tube having a first slot proximate the proximal end of the pull wire, the inner tube having a second outer diameter at the first slot, the second outer diameter of the inner tube being greater than the inner diameter of the outer tube, a portion of the outer circumferential surface of the inner tube adjacent to the first slot being in contact with and having a friction fit with the inner circumferential surface of the outer tube and is configured to prevent unintended movement of the inner tube and the pull wire with respect to the outer tube.

2. The delivery and detachment system of claim 1, wherein the first slot is spaced from the proximal end of the inner tube.

3. The delivery and detachment system of claim 1, wherein the first slot 44 extends to the proximal end of the inner tube.

4. The delivery and detachment system of claim 1, wherein the proximal end of the inner tube extends proximally beyond the proximal end of the outer tube.

5. The delivery and detachment system of claim 1, wherein the proximal end of the pull wire being fixedly connected to the proximal end of the inner tube.

6. The delivery and detachment system of claim 1, wherein the inner tube having a second slot proximate the proximal end of the pull wire, the second slot being approximately diametrically opposite of the first slot.

7. The delivery and detachment system of claim 1, wherein the pull wire distal end being releasably connected to an implantable intravascular treatment device.

8. The delivery and detachment system of claim 7, wherein the implantable intravascular treatment device is an embolic coil configured to treat an aneurysm.

9. A delivery and detachment system 10 for an implantable intravascular treatment device, the system comprising:
an outer tube comprising a proximal end, a distal end, an inner diameter, an inner circumferential surface, and a lumen extending axially therethrough from the proximal end to the distal end;
an inner tube comprising a proximal end, a distal end, a first outer diameter, an outer circumferential surface, and a lumen extending axially therethrough from the proximal end to the distal end, the inner tube being disposed radially within the outer tube at the proximal end of the outer tube, the inner diameter of the outer tube being greater than the outer diameter of the inner tube;
a pull wire positioned within the lumen of the outer tube and the lumen of the inner tube, the pull wire comprising a proximal end, and a distal end, the proximal end of the pull wire being positioned within the lumen of the inner tube, the proximal end of the pull wire being fixedly connected to the inner tube;
the inner tube having its proximal end portion disposed within the lumen of the outer tube being shaped to have at least one bend in the proximal end portion, the at least one bend being larger than the inner diameter of the outer tube such that a portion of the outer circumferential surface of the inner tube proximal end portion being in contact with and having a friction fit with the inner circumferential surface of the outer tube and is configured to prevent unintended movement of the inner tube and the pull wire with respect to the outer tube.

10. The delivery and detachment system of claim 9, wherein the proximal end of the inner tube extends proximally beyond the proximal end of the outer tube.

11. The delivery and detachment system of claim 9, wherein the proximal end of the pull wire being fixedly connected to the proximal end of the inner tube.

12. The delivery and detachment system of claim 9, wherein the inner tube proximal end portion has at least four bends and at least four axially spaced apart locations where the outer circumferential surface of the inner tube proximal end portion is in contact with and has a friction fit with the inner circumferential surface of the outer tube.

13. The delivery and detachment system of claim 12, wherein the inner tube proximal end portion has a sinusoidal shape.

14. The delivery and detachment system of claim 9, wherein the inner tube proximal end portion has a loop-the-loop shape.

15. The delivery and detachment system of claim 14, wherein the loop-the-loop shape ascends upward and curves back proximally on itself to form a complete loop, before descending back to its original distal direction.

16. The delivery and detachment system of claim 15, wherein the loop-the-loop shape includes at least two complete loops.

17. The delivery and detachment system of claim 16, wherein the loop-the-loop shape includes at least three complete loops.

18. The delivery and detachment system of claim 10, wherein the inner tube proximal end portion has a zigzag shape.

19. The delivery and detachment system of claim 18, wherein the zigzag shape extends distally and then bends back proximally at a first bend for a predetermined distance and then bends back distally at a second bend and then extends distally beyond the first bend.

20. The delivery and detachment system of claim 19, wherein the zigzag shape includes at least two zigzags.
